(19) United States
(12) Patent Application Publication
(10) Pub. No.: US 2009/0155216 A1
(43) Pub. Date: Jun. 18, 2009

(54) Grafting Material and Agent for Improvement in bone Quality

(75) Inventors: Yoichi Yamada, Nagoya-shi (JP); Minoru Ueda, Nagoya-shi (JP); Akihiro Yajima, Nagoya-shi (JP); Tomoyuki Kohgo, Nagoya-shi (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA UNIVERSITY, NAGOYA-SHI (JP)

(21) Appl. No.: 11/922,080

(22) PCT Filed: Jun. 13, 2006
(86) PCT No.: PCT/JP2006/311842

§ 371 Date: May 28, 2008

Related U.S. Application Data
(60) Provisional application No. 60/689,553, filed on Jun. 13, 2005.

(51) Int. Cl. A61K 45/00; A61P 19/00

(52) U.S. Cl. 424/93.3; 424/93.7

(57) ABSTRACT

The present invention has an object to provide a transplant material allowing systemic bone substance improvement to be realized by cell transplant. The present invention teaches to use a transplant material comprising a cell selected from the group consisting of an embryonic stem cell, a mesenchymal stem cell, an osteoblast cell, a preosteoblast, a chondrocyte and a cell having a capability of bone formation through systemic or topical administration for the improvement in bone quality.

### TECHNICAL FIELD

The present invention relates to transplant material and bone substance improving agent.

### BACKGROUND ART

Currently, Japan is an aging society in which an elder generation called baby-boom generation occupies 50% or more. Among them, class of patient affected by osteoporosis along with aging is on the rise. For instance, the number of patients of femoral neck fracture accompanied by bone substance decrease due to osteoporosis reaches approximately 100,000 annually.

As therapeutic methods for restoration or regeneration of such bone deficient sites due to osteoporosis or the like, autologous transplants using autologous tissues, for instance, fat, fascia, cartilage, bone fragment, or the like, may be cited. However, in the case of an autologous transplant, there is the problem that sufficient transplant materials with respect to the bone deficient site cannot be secured, or the problem of invading a healthy site. In addition, regarding allografts and xenografts, in addition to having the same problem of supply quantity as autologous transplants, the problems of immunity and viral infection are pointed out.

Meanwhile, in order to solve such problems, there are transplant materials that prosthetic joints made of titanium or the like by two types of cement or the like (cement type and porous type). In addition, a transplant material containing a mesenchymal stem cell (MSC) and platelet-rich plasma (PRP) is disclosed in the re-published Patent Publication No. WO2002/040071. In addition, osteoporosis does not occur only locally such as in femoral bones, and may occur systemically. As therapeutic methods applicable to systemic osteoporosis, in addition to therapeutic methods by drugs disclosed in Japanese Patent Application Laid-open No. 2003-55226 and Japanese Patent Application Laid-open No. 2004-43476, and the like, methods by diet, exercise or the like, are known generally.

### DISCLOSURE OF THE INVENTION

However, a preventive method or a therapeutic method that is effective against systemic bone density decrease or osteoporosis, or a preventive method or a therapeutic method that is effective against local bone disease, has not been found so far. In addition, a method that is effective in improving decreased local bone density by a non-local method has not been found. In particular, preventive/therapeutic methods that are effective on systemic osteoporosis are few, and according to the transplant materials mentioned above, which are local complements or for use in local regeneration, although local bone substance improvement is possible to some extent, they are not effective on systemic osteoporosis. In addition, in therapeutic methods using drugs, long term administration being required due to the nature of the disease, problems such as adverse effects may occur. In addition, methods by diet and exercise are no longer effective on severe osteoporosis patients.

Therefore, it is an object of the present invention to provide a transplant material allowing systemic or local bone substance improvement to be realized by cell transplant. In addition, it is another object of the present invention to provide a transplant material allowing local bone substance improvement to be realized by cell transplant without local administration.

Aiming for bone substance improvement, the present inventors examined systemic administration or local administration of mesenchymal stem cells and the like, and obtained the observations that, although being in a systemic administration mode, the decreasing tendency of systemic bone density could be remedied effectively, and at the same time, local bone density decrease could be suppressed definitely. In addition, they found that, also in a local administration, local bone density decrease could be suppressed effectively. That is to say, the following means are provided, according to the observations of the present inventors.

According to the present invention, a transplant material is provided, which contains one species or two or more species of cells selected from the group consisting of an embryonic stem cell, a mesenchymal stem cell, an osteoblast, a pre-osteoblast, a chondrocyte and a cell having osteogenic capability, and is used in bone substance improvement application by systemic administration or local administration. Note that, in the present invention, “bone substance improvement” means any of, osteogenesis, promotion of osteogenesis, increase in bone density, and prevention or treatment of bone disease.

Preferably, the transplant material of the present invention allows bone density increase to be promoted in a human or non-human bony part, and the cell is preferably a mesenchymal stem cell or a cell having osteogenic capability. In addition, the cell is preferably an autologous cell. Also, the transplant material of the present invention is preferably for intravascular administration or for intravenous injection, and in addition, the transplant material of the present invention is preferably for administration into bone medullary cavity. Furthermore, the transplant material of the present invention can be made to be for the prevention or for the treatment of bone disease and is useful for systemic bone disease and for localized bone disease; particular in the case of systemic administration mode, it is useful in systemic bone disease. In addition, the transplant material of the present invention can be any of orthopedic surgery use, aesthetic plastic surgery use, plastic surgery use, dentistry use and oral surgery use. In addition, the transplant material of the present invention may be for use in the treatment against spinal cord damage in plastic surgery or the like, or may be for use in the treatment of jawbone extension in oral surgery.

According to the present invention, a bone substance improving agent containing the transplant material described in any of the above, which administration mode is systemic administration or local administration, is provided. The systemic administration mode is preferably intravascular administration. In addition, the local administration mode is preferably administration into bone medullary cavity.

According to the present invention, a production method is provided for producing the transplant material described in any of the above, comprising a step of preparing one species or two or more species of cells selected from the group consisting of an embryonic stem cell, a mesenchymal stem cell, an osteoblast, a pre-osteoblast, a chondrocyte and a cell having osteogenic capability. In the production method for the transplant material of the present invention, the cell is preferably an autologous cell, and in addition, the method comprises a step of culturing one species or two or more species of cells selected from the group consisting of an embryonic stem cell, a mesenchymal stem cell, an osteoblast, a pre-osteoblast, chondrocyte and a cell having osteogenic capability is preferably provided. In addition, a step of conserving one species or two or more species of cells selected from the group consisting of an embryonic stem cell, a mesenchymal stem cell, an osteoblast, a pre-osteoblast, a chondrocyte and a cell having osteogenic capability is preferably provided. In addition, according to the present invention, a production method for a bone substance improving agent provided with a step of preparing any of the transplant materials mentioned above is also provided.

According to the present invention, a method is provided, which is a bone substance improvement method comprising the step of systemically administering or locally administering any of the transplant materials described above. In the case of systemic administration, the administration step is preferably vascular administration, such as, parenteral administration. In addition, in the case of local administration circumstance, administration is into bone medullary cavity. In addition, systemic administration and local administration may be used in combination. That is to say, the step may be made to administer the transplant material of the present invention locally to the region requiring bone substance improvement, or to administer the transplant material of the present invention systemically, prior to the systemic administration or local administration step, or simultaneously to the systemic administration step or local administration step, or after the systemic administration step or local administration step. Note that a constituent other than a cell, such as, scaffold, PRP, PRP gel, various growth factors, ECM protein, gelation material and thickener may be administered alone to the region, or these and a cell may be administered locally.

### BRIEF DESCRIPTION OF THE DRAWINGS

FIG. 1 is a figure showing an administration mode of a mesenchymal stem cell in an example;

FIG. 2 is a figure showing the bone density measurement result for a systemic administration group of an example;

FIG. 3 is a figure showing the result of a μCT photograph in an example; and

FIG. 4 is a figure showing the bone density measurement result for a local administration group of an example.

### BEST MODE FOR CARRYING OUT THE INVENTION

The present invention relates to a transplant material containing one species or two or more species of cells selected from the group consisting of an embryonic stem cell, a mesenchymal stem cell, an osteoblast, a pre-osteoblast, a chondrocyte and a cell having osteogenic capability, and to its use in a bone substance improvement application by systemic administration or local administration, in addition, it relates to a bone substance improving agent containing the transplant material, production of the transplant material or bone substance improving agent as well as a bone substance improvement method using the transplant material or bone substance improving agent, and in particular, a method for the prevention or treatment of bone disease.

By administering systemically such as into vascular system administration or by administering locally such as into bone medullary cavity, the transplant material of the present invention, a rise in bone density, that is to say, a regeneration of bone tissue is possible, at a site with decreased bone density. Therefore, according to the transplant material of the present invention, improvement of systemic or local bone density, or the like, is possible without recourse to local administration. In addition, from the fact that bone substance improvement is possible without recourse to local administration, combined use is also possible with locally administered other transplant material, drug, or the like, aimed at bone substance improvement. In addition, bone substance can be improved effectively by administering locally a scaffold separately to the site requiring an improvement in bone substance, or the like. In addition, a rise in bone density and regeneration bone tissue is also possible by local administration of the transplant material of the present invention. Therefore, the transplant material of according to the present invention allows systemic administration and local administration to be carried out in combination. In addition, complementary or additive bone substance decrease prevention or bone substance improvement becomes possible, with minimal invasiveness, and furthermore, timely, by regenerative medicine using such a transplant material for use in bone substance improvement. Hereinafter, the present invention will be described in detail.

(Transplant Material)

(Cell Used As Transplant Material)

The transplant material of the present invention contains a cell selected from the group consisting of an embryonic stem cell, a mesenchymal stem cell, an osteoblast, a pre-osteoblast, chondrocyte and a cell having osteogenic capability. The cell used in the transplant material of the present invention may be only one species among these, or may be two or more species.

Embryonic stem cell and/or mesenchymal stem cell are used preferably for systemic administration use. More desirable is mesenchymal stem cell. Such a cell may be from these cell sources and collected from a human or non-human animal, and may have been further cultured. Alternatively, it may be one that has been established by an artificial method. Although the cell may be a heterologous cell as long as rejection is effectively avoided, preferably, it is a homologous cell, and more preferably, an autologous cell is used. In addition, the cell may have been genetically modified as necessary. In addition, it may also be one that has been cryopreserved.

For instance, mesenchymal stem cell can be collected with iliac bone marrow, jawbone bone marrow, peripheral blood, dental pulp, periosteum, umbilical cord blood, or the like, as a supply source. Methods for collecting mesenchymal stem cells from these supply sources are well known to those skilled in the art. The collected mesenchymal stem cell can be used as-is, or may be cultured to obtain the quantity required. In addition, methods for culturing mesenchymal stem cells are described in Boo, J. S., Yamada, Y., Hibino, Y., Okazaki, Y., Okada K., Hata, K., Yoshikawa, T., Sugiura Y., and Ueda, M. Tissue-Engineered Bone Using Mesenchymal Stem Cells and a Biodegradable scaffold. J. Craniofac. surg. 13, 231-239, 2002, Yamada, Y., Boo, J. S., Ozawa, R., Nagasaka, T., Okazaki, Y., Hata, K., and Ueda, M. Bone regeneration following injection of mesenchymal stem cells and fibrin glue with a biodegradable scaffold. J. Cranio-maxillofac. surg. 31, 27-33, 2003, and the like, in addition to being well known to those skilled in the art.

In addition, cell having osteogenic capability is preferred for local administration use. A cell having osteogenic capability means cell that may form a bone tissue containing, osteoblast, osteoblast, pre-osteoblast, mesenchymal stem cell and ES cell differentiated into a bone system cell, and the like. Preferably, mesenchymal stem cell differentiated into bone system cell is used. Note that “differentiated into bone system cell” means a state in which a mesenchymal stem cell or an embryonic stem cell in undifferentiated state has been directed toward bone system cell. Although the mesenchymal stem cell and ES cell [there is be a mistake in the Japanese source] differentiated into bone system is preferably an autologous cell, it may be a homologous, allologous cell, and in addition, human derived cell can be exploited. Osteoblast, pre-osteoblast and chondrocyte can be collected from iliac bone marrow, jawbone bone marrow, peripheral blood, dental pulp, periosteal or umbilical cord blood. In addition, a mesenchymal stem cell differentiated into a bone system cell can be prepared, for instance, by culturing a collected mesenchymal stem cell in vitro, under conditions where differentiation into bone system cell is induced. In order to induce the differentiation into bone system cell, for example, culture medium containing β-glycerophosphate, dexamethasone and L-ascorbic acid can be cited. Note that the culture condition is not limited to this, and differentiation induction conditions for bone system cell well know in prior art can also be exploited. The mesenchymal stem cell differentiated into bone system cell may be one that has been freeze processed. In addition, as supply source of undifferentiated mesenchymal stem cell, collection is possible from bone marrow, such as, iliac bone marrow and jawbone bone marrow, peripheral blood, dental pulp, periosteal or umbilical cord blood. After collection therefrom by methods well know in the prior art, mesenchymal stem cells are selected based on the presence or the absence of an adhesion property thereof. That is to say, undifferentiated mesenchymal stem cells can be obtained by selecting among the cells contained in bone marrow, or the like, those having adhesion property.

(Constituents Other Than Cell)

Whether it is for systemic administration use or for local administration use, it is desirable that the transplant material of the present invention adopts the form of a suspension, in which cells have been suspended in a suitable medium. As such medium, physiological saline, suitable buffering solution and the like can be selected, although there is no particular limitation. The transplant material of the present invention may contain blood or a portion thereof as medium. For example, as portion of blood (constituent), blood plasma, platelet-rich plasma (PRP), and the like, can be used. PRP conforms, for instance, to product name: Platelet Concentrate “Nisseki” (manufactured by Japanese Red Cross Society). PRP can be prepared by treating collected blood by centrifugal separation, or the like. Note that PRP is preferably self-derived PRP. Note that PRP may be administered separately from the transplant material of the present invention, without being included in the transplant material of the present invention.

In addition, the transplant material of the present invention can contain a growth factor. For instance, platelet derived growth factor (PDGF), transforming growth factor β (TGF-β1, TGF-β2), vascular endothelial growth factor (VEGF), EGF, insulin-like growth factor (IGF)-I, basic fibroblastic growth factor (b-FGF), bone-derived factor (BMP), and the like, which are growth factors contained in platelet-rich plasma, can be included. In particular, the combined use with PRP or one species or two or more species of growth factors allows the bone density to be enhanced effectively at the site where osteogenesis or promotion of osteogenesis is to be carried out. Note that a growth factor may be administered separately from the transplant material of the present invention, without being included in the transplant material of the present invention.

When using the transplant material of the present invention for local administration use, it may be locally administered alone (with physiological saline, or the like as medium), or may contain, in addition to PRP and various growth factors mentioned above, one species or two or more species selected from scaffold, ECM protein, gelation material and thickener. For instance, when cell alone is administered locally using physiological saline or the like, cells can reach the entirety of bone marrow, while when a scaffold is used, cells can be confined to a given region of the bone, and improve the bone substance of the required portion only.

As such scaffolds, in addition to various artificial bones and biological bones (including autologous bone, and the like), polymer materials that are decomposed and absorbed in vivo may be cited. As such polymer materials, for instance, synthetic polymer molecules, such as, polylactic acid, polyglycolic acid, copolymer of lactic acid and glycolic acid, poly-ε-caprolactone, copolymer of ε-caprolactone and lactic acid or glycolic acid, polycitric acid, polymalic acid, poly-α-cyanoacrylate, poly-β-hydroxybutyric acid, polytrimethylene oxalate, polytetramethylene oxalate, polyorthoester, polyorthocarbonate, polyethylene carbonate, polypropylene carbonate, poly-γ-benzyl-L-glutamate, poly-γ-methyl-L-glutamate and poly-L-alanine, polysaccharides, such as, starch, alginic acid, hyaluronic acid, chitin, pectic acid and derivatives thereof, or gelatin, collagen (the type of collagen and the extraction method thereof may be any) and the like, may be cited.

In addition, PRP gel, which is blood platelet and fibrinogen contained in PRP gelled using thrombin or the like, can also be used as scaffold. PRP gel can function as an effective scaffold for osteogenesis by conjugating to the polymer molecular material described above. In addition, PRP gel builds a scaffold that is also satisfactory from the point that it contains various growth factors as described above.

In addition, as another constituent that can be supplied to bone substance improvement site, extracellular matrix (ECM) protein may be cited. Although ECM is preferably autologous, it may also be homologous allologous.

Gelation material can contain, for instance, thrombin and calcium chloride. By locally administering such a gelation material, thrombin acts on the fibrinogen in PRP, generating fibrin; then, the viscosity increases by the coagulation action of fibrin. Gelation materials may be those that act on a constituent in PRP and increase viscosity in this way, or those that exert per se a viscosity increasing effect may be used. In addition, a second gelation material may be used, which is added to the gelation material and works after transplant, modifying the fluidity (viscosity) of the transplant material of the present invention. By having a suitable degree of fluidity at the time of use and then increasing the viscosity after the transplant, the fixability at the application site rises, and osteogenesis becomes effective. As the gelation material, collagen and fibrin pastes may be cited. In addition, as thickener, polysaccharide thickener such as sodium alginate, and thickeners such as glycerin and vaseline may be cited.

(Administration Mode of the Transplant Material)

The transplant material of the present invention may adopt the mode of systemic administration or local administration. Although there are no particular limitations, from the point of view of systemic administration intended for cell transplant having bone tissue as target, the mode of systemic administration is preferably vascular administration via blood vessel or lymph vessel. Among them, intravascularly administering vein, and the like, is preferred. As administration method, injection, drip infusion, catheter and the like can be used.

In addition, although there are also no particular limitations regarding local administration, administering into bone medullary cavity or the like is possible. As administration method, injection, drip infusion, catheter and the like can be used.

For administration of the transplant material of the present invention, PRP, growth factor, and the like, which are constituents other than the cells described above, can be combined and administered. Note that such constituents other than cells may be included in the transplant material along with the cells, or may be ones that are administered separately from the transplant material. For systemic administration, when administering intravascularly, or the like, the transplant material of the present invention, it is suitably prepared with an extent of liquidity (viscosity, liquidity and the like) allowing for injection into blood vessel or the like with a catheter or a syringe. In addition, for local administration, it is suitably prepared with an extent of liquidity allowing for injection into bone medullary cavity with a syringe or a catheter. Note that in order to formulate the transplant material as an injectable, a transfusion agent or the like, various carriers, additives and the like, which are pharmacologically allowed, can be used.

Although the number of cells in a cell suspension to be administered systemically can be 1×10⁵ cell/ml or greater, it is preferably 1×10⁶ cell/ml or greater but 1×10⁸ cell/ml or less. In addition, the same number of cells is possible for local administration as well.

(Combined use of systemic administration and individual local administration of another constituent such as scaffold)

When administering systemically the transplant material of the present invention, prior to administration (transplant) of the transplant material or along with the transplant, scaffold, PRP, various growth factors, ECM protein, gelation material, thickener and the like can be supplied to a bone substance improvement site (a site requiring improvement of bone substance, idem hereinafter), such as, a site where osteogenesis or promotion of osteogenesis is to be carried out (bone disease site, site of treatment or prevention in orthopedic surgery, site of treatment or prevention in aesthetic plastic surgery, site of treatment or prevention in plastic surgery, site of treatment or prevention in dentistry, site of treatment or prevention in oral surgery, and the like). In this way, implantation at the bone disease site or the like, or cell proliferation or the like at the bone disease site, of the transplanted cell is promoted, allowing osteogenic (regeneration) at the bone disease site to be promoted. As such scaffold, gelation material and thickener, those already given as examples of those used at local administration time can be used.

(Combined use of systemic administration and local administration of the transplant material of the present invention)

As the transplant material of the present invention is to be administered systemically, local administration of the transplant material of the present invention to the bone substance improvement site may be used in combination along with administering systemically the transplant material of the present invention. For instance, along with administering a mesenchymal stem cell into a vein, cell having osteogenic capability may be administered locally by bone medullary cavity injection or the like. The combined use of local administration and systemic administration allows a synergistic effect to be anticipated, along with allowing locally focused bone substance improvement to be anticipated.

The transplant material of the present invention may be used in combination with a treatment with an artificial construct, such as, prior art implantable artificial bone or articulation, and systemically administered. In this way, implantation of the artificial construct can be promoted.

(Application of the Transplant Material)

The various modes of transplant material of the present invention described above can be used for bone substance improvement. That is to say, typically, they can be exploited in, the suppression of a decrease in, the maintenance of and the increase in, bone density, and the treatment, prevention and remedy, of various bone diseases, by osteogenesis or osteogenesis promotion. In addition, the transplant material of the present invention can also be applied in the realm of orthopedic surgery, aesthetic plastic surgery, plastic surgery, dentistry and oral surgery. For instance, it can be used in the realm of orthopedic surgery, for regeneration, bone extension, or the like, of bone/articular cartilage of hip joint, bone head, knee joint and the like, in the realm of aesthetic plastic surgery, for rhinoplasty and wrinkle removal by cartilage regeneration, in the realm of dentistry and oral surgery, for reinforcement or reconstruction of bone substance of the jawbone for anchorage of an implant (artificial tooth root), enhancement/regeneration of alveolar bone, reconstruction of jawbone after surgery, reconstruction of the cleft jaw portion of a cleft lip, jaw and palate, and the like. In addition, in the realm of orthopedic surgery, it can also be used for treatment against spinal cord damage, and in the realm of oral surgery, it can also be used for jawbone extension.

The transplant material of the present invention is preferably used for bone substance improvement or prevention/treatment in bone diseases of human and non-human animals (preferably human). The bone diseases are diseases in which symptoms such as a decrease in bone density and deterioration of bone tissue is involved, or the like, accompanied by a decrease in the amount of bone. As such bone diseases, (1) primary osteoporosis (primary osteoporosis accompanying ageing, osteoporosis accompanying menopause, osteoporosis accompanying oophorectomy, and the like), (2) secondary osteoporosis (for instance, glucocorticoid induced osteoporosis, hyperthyroid osteoporosis, osteoporosis due to kidney failure, inflammatory osteoporosis, osteoporosis accompanying Cushing's syndrome rheumatoid osteoporosis, or the like), (3) bone metastasis, hypercalcaemia, Paget's disease, bone defect (alveolar bone defect, mandibular bone defect, childhood idiopathic bone defect, and the like), osteonecrosis, and the like may be cited.

(Transplant Material Production Method)

The method for producing the transplant material of the present invention can comprise the step of preparing a cell used in the transplant material of the present invention. That is to say, the method can be provided with the step of collecting or establishing any of the cells described above, and can be provided additionally with the step of culturing the cells. In addition, a step of inducing differentiation may be implemented. Regarding differentiation induction, it is as already described. In addition, the method for producing the transplant material of the present invention can comprise a step of conserving any of the cells described above. Once acquired, by conserving the cells until the time of use, the transplant can be carried out timely. Regarding the conservation step, the cells are preferably frozen prior to conservation, and in addition, a cell preservative can be added when freezing.

(Bone Substance Improving Agent and Bone Disease Prevention/Treatment Agent)

The bone substance improving agent and the bone disease prevention/treatment agent (hereinafter, simply bone improving agent and the like) of the present invention contain the transplant material of the present invention. The bone substance improving agent and the like of the present invention is used by systemic administration and/or local administration in bone substance improvement applications and applications for bone disease prevention/treatment. The bone substance improving agent and the like of the present invention can be used in the same applications as the transplant material of the present invention by the same methods. In particular, as the bone substance improving agent and the like of the present invention, mode formulated for systemic administration use such as for intravascular administration and mode formulated for local administration use such as for administration into bone medullary cavity may be given. Such bone substance improving agent and the like may contain estrogen, vitamin K, bis-phosphonate, or the like, which are well-known for bone substance improvement, in addition, may be ones that are used in combination and administered. Note that the bone substance improving agent of the present invention can be acquired via the transplant material of the present invention, by using a well-known pharmaceutical carrier or the like.

(Bone substance improvement method and bone disease prevention/treatment method) The bone substance improvement method and bone disease prevention/treatment method of the present invention comprise the step of administering systemically and/or administering locally the transplant material or the bone substance improving agent and the like of the present invention. Bone substance improvement as well as prevention or treatment of bone disease become possible by using the transplant material of the present invention or the like, as already described. Note that, as already described in the administration mode of transplant material, prior to systemically administering the transplant material of the present invention or the like, or along with systemic administration, local administration of scaffold, growth factor, PRP, ECM protein, gelation material, thickener, the transplant material of the present invention (including the mode of administering a cell alone or a scaffold, or the like, simultaneously or separately) and other transplant materials, introduction of an artificial construct, and the like, can also be carried out.

### EXAMPLES

Hereinafter, the present invention will be described concretely, giving examples. However, the examples are not limiting the present invention.

In the examples, osteoporosis model rats were created to verify that the transplant material of the present invention can remedy bone density decrease.

(Creation of Osteoporosis Model Rat)

Osteoporosis model rat was established by extracting ovaries. These methods were carried out according to Hitoshi Saino et al., J Bone Miner Res 1997; 12:1844-1850. Prior to cell transplant described below, the rat after treatment was measured for bone density (using a CT apparatus called DXA (dual energy X-ray absorptiometer)) to verify that an osteoporosis model was established.

(Collection and Culture of Mesenchymal Stem Cell)

Mesenchymal stem cells (hereinafter abbreviated as MSCs) collected from the femoral bone marrow fluid of a GFP rat were separated and cultured, and a culture was carried out until a target number of cells. The culture methods were carried out according to Boo, J. S., Yamada, Y., Hibino, Y., Okazaki, Y., Okada K., Hata, K., Yoshikawa, T., Sugiura Y., and Ueda, M. Tissue-Engineered Bone Using Mesenchymal Stem Cells and a Biodegradable scaffold. J. Craniofac. surg. 13, 231-239, 2002, Yamada, Y., Boo, J. S., Ozawa, R., Nagasaka, T., Okazaki, Y., Hata, K., and Ueda, M. Bone regeneration following injection of mesenchymal stem cells and fibrin glue with a biodegradable scaffold. J. Cranio-maxillofac. surg. 31, 27-33, 2003. For GFP rat, one developed by Pr. Okabe et al. from Osaka University was purchased through Japan SLC. This rat has a GFP gene incorporated in all tissues, allowing the behavior of a transplant cell to be tracked. This rat is described in Takahito Ito et al., J. Am Soc Nephrol 12: 2625-2635, 2001.

(Transvenous Systemic Administration of Mesenchymal Stem Cell)

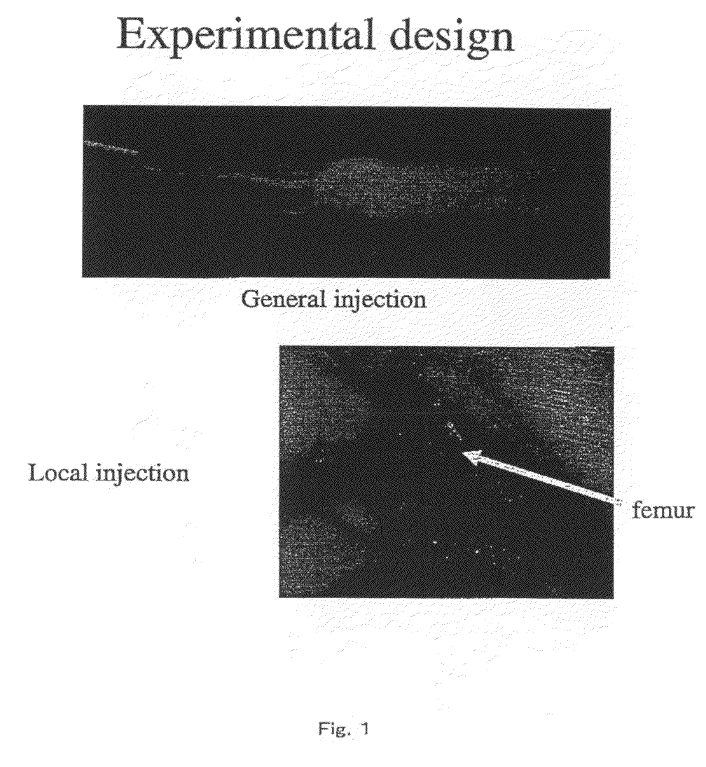

Cultured MSCs were suspended with physiological saline to prepare cell suspensions with number of cells of 5×10⁶ cells/ml and 1×10⁷ cells/ml. Each cell suspension was injected through the tail vein of a model rat (nude rat) for which establishment of osteoporosis model was verified (refer to FIG. 1). Note that bone density measurements for establishing osteoporosis model were on the order of two weeks, one month, two months and three months after oophorectomy.

(Preparation of Mesenchymal Stem Cell Differentiated Into Bone System Cell)

Undifferentiated mesenchymal stem cell was collected from the femur of the above GFP rat, and differentiation was induced to prepare a mesenchymal stem cell differentiated into bone system cell.

Methods for inducing differentiation of mesenchymal stem cell was according to Boo, J. S., Yamada, Y., Hibino, Y., Okazaki, Y., Okada K., Hata, K., Yoshikawa, T., Sugiura Y., and Ueda, M. Tissue-Engineered Bone Using Mesenchymal Stem Cells and a Biodegradable scaffold. J. Craniofac. surg. 13, 231-239, 2002, Yamada, Y., Boo, J. S., Ozawa, R., Nagasaka, T., Okazaki, Y., Hata, K., and Ueda, M. Bone regeneration following injection of mesenchymal stem cells and fibrin glue with a biodegradable scaffold. J. Cranio-maxillofac. surg. 31, 27-33, 2003, Yamada, Y., Ueda, M., Naiki, T., Takahashi, M., Hata, K., and Nagasaka, T. Autogenous injectable Bone for Regeneration with Mesenchymal Stem Cells (MSCs) and Platelet-Rich Plasma (PRP)—Tissue-engineered bone regeneration—Tissue Eng. 10 (5/6), 955-964, 2004. That is to say, bone marrow containing undifferentiated mesenchymal stem cell was collected by bone marrow puncturing of the femur of the above GFP rat, bone marrow cell was cultured in essential medium, low glucose DMEM, growth supplement (Manufactured by Cambrex Corporation), and differentiation of mesenchymal stem cell into bone system cell was induced by three supplements (dexamethasone, sodium β-glycerophosphate, and L-ascorbic acid 2-phosphate). Mesenchymal stem cells differentiated into bone system cells were identified by detecting alkaline phosphatase activity using p-nitrophenyl phosphatase as substrate. The mesenchymal stem cells were treated with trypsin prior to being used in transplant.

(Local Administration)

Mesenchymal stem cells prepared as described above were injected into the femur of a rat (nude rat) along with physiological saline or PRP, using a bone marrow puncture needle, as shown in FIG. 1. The numbers of cells transplanted were 5×10⁶ cells/ml and 1×10⁷ cells/ml.

(Preparation of PRP)

For PRP, whole blood was collected from rat peripheral blood, after a 5 minute, 1100 rpm centrifugation, yellow plasma (containing buffy coat along with blood platelets and white blood cells) was recovered with a long cannula to a neutral monobed, and blood platelets were prepared as a single pellet by a 10 minute, 2500 rpm centrifugation. The above PRP was resuspended in residual blood plasma and used for gelation of PRP. Gelation of PRP was carried out by adding a thrombin/calcium chloride solution to the above PRP and mixing while including bubbles. The above thrombin/calcium chloride solution was prepared by dissolving 10,000 U of bovine thrombin in lOml of 10% calcium chloride solution.

(Evaluation)

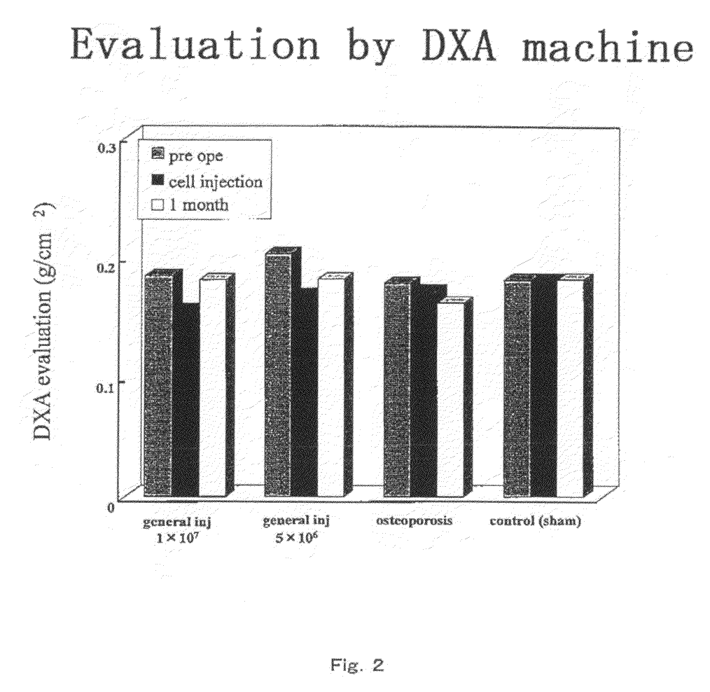

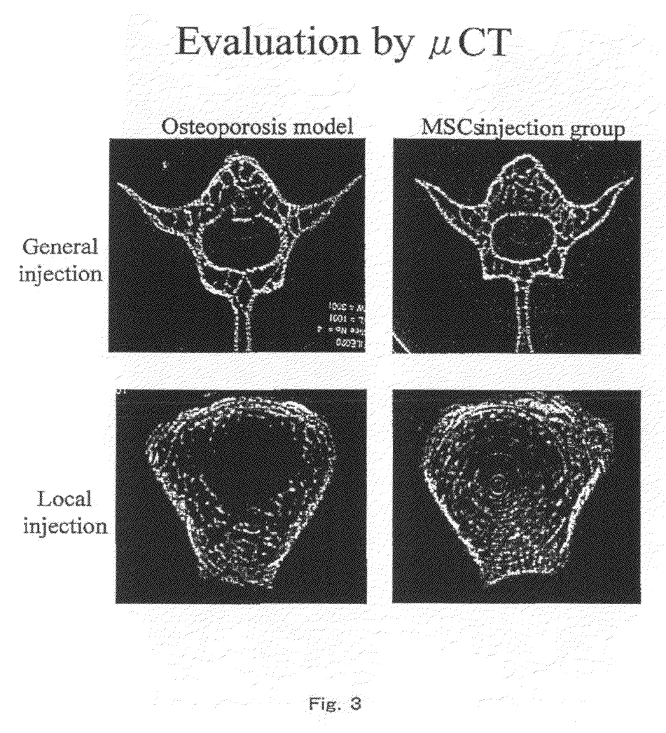

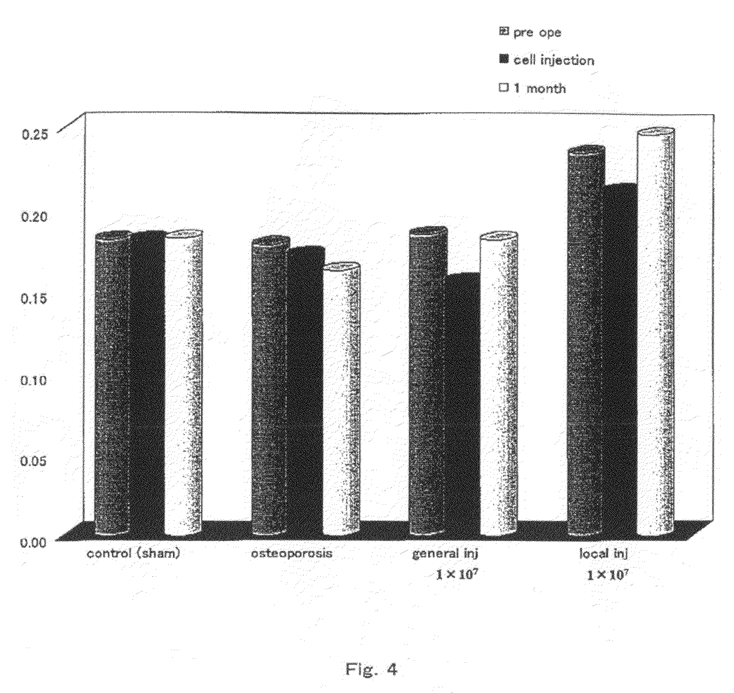

Immediately after transplanting cells into an osteoporosis model rat by a given method, one month after the transplant, and after two months and three months have elapsed, similarly, the bone density was measured by the same methods as bone density measurement for verifying the establishment of osteoporosis model rat, and based on the bone density, the status of cell transplant was determined. Results of measurement of bone density in the systemic administration group prior to transplant, at transplant time, and one month after transplant, are shown in FIG. 2. In addition, the animals were sacrificed in a state where improvement of bone density was obtained, and μCT were taken. In the systemic administration group, the spine was imaged, and in the local administration group, the femur was imaged. The μCT results are shown in FIG. 3. In addition, results of bone density measurement in the local administration group prior to transplant, at transplant time, and one month after transplant are shown in FIG. 4.

Regarding the systemic administration group, as shown in FIG. 2 and FIG. 3, although no change in the bone density value was observed in the control group, a decrease in the bone density value was observed in the oophorectomy (osteoporosis control) group, and an osteoporosis model was established. In addition, although a decreasing tendency of the bone density value was observed in the cell transplant group until cell transplant time, a rise in the bone density value was observed by carrying out cell transplant. In particular, on the order of 10% increase in bone density was observed in the 1×10⁷ cells/ml treatment group at one month after transplant when compared to control immediately after transplant [Japanese source is suspect]. In addition, improvement of bone density was also observed in μCT. Regarding the number of cells, 1×10⁷ was more effective. Considering that an increase of bone density by prior art drug treatment is on the order of 3 to 4%, it was found that bone density improvement effect by the transplant material of the present invention was excellent.

In addition, regarding the local administration group, in the cell transplant group, a rise in the bone density value was observed while at the same time, improvement of bone density was also observed in ACT, as shown in FIG. 3 and FIG. 4. An increase of on the order of 10% was observed in bone density after cell transplant in comparison with control. Therefore, similarly to when administered systemically, the transplant material of the present invention was found to have excellent bone density improvement effect also when administered locally.

From the foregoing, improvement of bone density was observed by systemically administering a mesenchymal stem cell against osteoporosis. From this, it was found that, according to the transplant material of the present invention, treatment or prevention could be carried out, which was effective against osteoporosis occurring systemically, and in addition, against cases that could be addressed so far only locally. A clear effect of bone density decrease suppression or bone density increase could be obtained by systemic administration, also in localized regions such as spinal cord.

In addition, improvement of bone density was observed by locally administering a mesenchymal stem cell differentiated into bone system cell against osteoporosis. From this, the transplant material of the present invention was found to be effective in local treatment of osteoporosis occurring systemically. In addition, the transplant material of the present invention was found to be also effective with respect to local bone diseases and bone substance improvement site.

Note that the contents of the references quoted herein are incorporated by quotation in their entirety. In addition, the present application has provisional U.S. patent application No. 60/689,553 filed on 13 Jun. 2005 as the basis of claim of priority, the contents thereof being incorporated herein by quotation in its entirety.

### INDUSTRIAL APPLICABILITY

The transplant material of the present invention is useful in the prevention/treatment of bone diseases such as osteoporosis, in addition to the field of bone regenerative medicine, for instance, in orthopedic surgery, cosmetic surgery, dentistry, oral surgery, otorhinolaryngology and the like, and bone substance improvement, such as, suppression of the decrease of, maintenance of and increase in bone density.

1-35. (canceled)

36. A method for improving of bone substance compridsing steps;
transplanting a material containing one species or two or more species of autologous cells selected from the group consisting of an embryonic stem cell, a mesenchymal stem cell, an osteoblast, a pre-osteoblast, a chondrocyte and a cell having osteogenic capability by systemic administration.

37. The method according to claim 36, wherein bone density increase is promoted in a human or non-human bone part.

38. The method according to claim 36, wherein said cell is a mesenchymal stem cell or a cell having osteogenic capability.

39. The method according to claim 36, wherein said autologous cell is a cultured autologous cell.

40. The method according to claim 36, for vascular administration or for intravenous administration.

41. The method according to claim 36, for use in the prevention or for use in the treatment of a bone disease.

42. The method according to claim 41, wherein said bone disease is generalized or localized.

43. The method according to claim 41, wherein said bone disease is any selected from osteoarthritis, lumbar back pain and chronic rheumatoid arthritis.

44. The method according to claim 41, wherein said bone disease is selected from bone defect, osteoporosis, bone fracture and periodontal disease.

45. The method according to claim 41, wherein said bone disease is either of Paget's disease of bone and Parkinson's disease.

46. The method according to claim 41, wherein said bone disease is either of osteopenia and osteomalacia.

47. The method according to claim 41, wherein said osteoporosis is menopausal osteoporosis.

48. The method according to claim 36, for any of orthopedic surgery use, cosmetic surgery use, plastic surgery use, dentistry use and oral surgery use.

49. A production method for a transplant material for use in bone substance improvement in systemic administration use, the method comprising a step of preparing one species or two or more species of autologous cells selected from the group consisting of an embryonic stem cell, a mesenchymal stem cell, an osteoblast, a pre-osteoblast, a chondrocyte and a cell having osteogenic capability.

50. The production method according to claim 49, wherein said autologous cell is a cultured cell.

51. The production method according to claim 49, comprising a step of conserving one species or two or more species of cells selected from the group consisting of an embryonic stem cell, a mesenchymal stem cell, an osteoblast, a pre-osteoblast, chondrocyte and a cell having osteogenic capability.
